(19) United States
(12) Patent Application Publication
(10) Pub. No.: US 2024/0343675 A1
(43) Pub. Date: Oct. 17, 2024

(54) AN IMPROVED PROCESS FOR THE PREPARATION OF N-ETHYL- A -METHYL-3-(TRIFLUOROMETHYL) PHENETHYLAMINE HYDROCHLORIDE

(71) Applicant: BIOPHORE INDIA PHARMACEUTICALS PVT. LTD., Hyderabad (IN)

(72) Inventors: Manik Reddy PULLAGURLA, Hyderabad (IN); Bhaskar Reddy PITTA, Hyderabad (IN); Jagadeesh Babu RANGISETTY, Hyderabad (IN)

(21) Appl. No.: 18/684,974

(22) PCT Filed: Aug. 19, 2022
(86) PCT No.: PCT/IB2022/057782

§ 371 Date: Feb. 20, 2024

(30) Foreign Application Priority Data
Aug. 19, 2021 (IN) 202141037571

(51) Int. Cl. C07C 211/29

(52) U.S. Cl. CPC C07C 211/29

(57) ABSTRACT

An improved process for the preparation of N-ethyl-α-methyl-3-(trifluoromethyl) phenethylamine hydrochloride is provided, having purity greater than 99.5% by HPLC using highly pure 3-(trifluoromethyl) aniline hydrochloride as a key starting material. The disclosed provides process for the purification of N-ethyl-α-methyl-3-(trifluoromethyl) phenethylamine hydrochloride, which is substantially free of Impurity A and Impurity B.

### CROSS-REFERENCE TO RELATED APPLICATIONS

This application claims priority to PCT Application No. PCT/IB2022/057782, having a filing date of Aug. 19, 2022, which is based on IN 202141037571, having a filing date of Aug. 19, 2021, the entire contents both of which are hereby incorporated by reference.

### FIELD OF TECHNOLOGY

The following relates to an improved process for the preparation of N-ethyl-α-methyl-3-(trifluoromethyl) phenethylamine hydrochloride (1), having purity greater than 99.5% by HPLC using highly pure 3-(trifluoromethyl) aniline hydrochloride (5) as a key starting material.

### BACKGROUND

N-ethyl-α-methyl-3-(trifluoromethyl) phenethylamine hydrochloride is known as Fenfluramine hydrochloride (1). Fenfluramine hydrochloride is marketed under the brand name FINTEPLA® and structurally known as compound of Formula I:

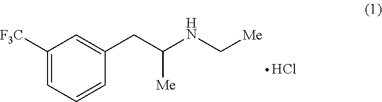 [SMILES: CCNC(C)Cc1cccc(C(F)(F)F)c1 ; Cl]

Fenfluramine is a schedule IV-controlled substance used for the treatment of seizures associated with Dravet syndrome. It is a white to off-white crystalline solid and marketed as oral solution.

There are several conventional art processes disclosing process for the preparation of Fenfluramine which are hereby incorporated as reference in their entirety.

Bulletin de la Société Chimique de France, 1993, Vol 130, pg 450-458 discloses preparation of Fenfluramine (1) using 1-bromo-3-(trifluoromethyl)benzene in presence of cupric bromide and magnesium turnings.

U.S. Pat. No. 5,811,586 A discloses a process for the preparation of 1-(3-trifluoromethyl) phenyl-propan-2-one by diazotizing reaction of m-trifluoromethylaniline in presence of copper salt.

U.S. Pat. No. 5,587,398 A discloses process for the preparation of norfenfluramine using 3-trifluoromethylphenylacetone in presence of ammonium acetate and sodium cyanoborohydride and further purified by vacuum distillation or through the bisulfite complex to obtain the pure product.

U.S. Pat. No. 10,947,183 B2 reported a process for the preparation of fenfluramine, wherein the fenfluramine has less than 0.2% by weight of 4-fenfluramine or a salt thereof.

Fenfluramine obtained according to the conventional art process produces impurities such acetate impurity, dimer impurity, fenfluramine Regio isomers, fenfluramine Alcohol and norfenfluramine. Hence, the inventors of the present disclosure have developed a process for the preparation of fenfluramine hydrochloride, which is industrially feasible, economical and devoid of fenfluramine Regio isomers impurities to obtain a product having purity greater than 99.5% by HPLC.

### SUMMARY

An aspect relates to an improved process for the preparation of N-ethyl-α-methyl-3-(trifluoromethyl) phenethylamine hydrochloride (1), having purity greater than 99.5% by HPLC using highly pure 3-(trifluoromethyl) aniline hydrochloride (5) as a key starting material.

The second aspect of embodiments of the present invention provides process for the purification of N-ethyl-α-methyl-3-(trifluoromethyl) phenethylamine hydrochloride (1), which is substantially free of Impurity A and Impurity B.

The third aspect of embodiments of the present invention provides industrially viable process for the preparation of 3-(trifluoromethyl) aniline hydrochloride (5) having purity greater than 99.5% by HPLC.

The fourth aspect of embodiments of the present invention provides process for the purification of 3-(trifluoromethyl) aniline hydrochloride (5), which is substantially free of Impurity C and Impurity D.

Accordingly, in one aspect, embodiments of the present invention provides an improved process for the preparation of Fenfluramine hydrochloride (1) having purity greater than 99.5% by HPLC using highly pure 3-(trifluoromethyl) aniline hydrochloride (5) as a key starting material. which comprises:
i. reacting 3-trifluoromethylaniline hydrochloride (5) with sodium nitrite or nitrous acid to obtain 1-chloro-2-(3-(trifluoromethyl) phenyl) diazene (4);
ii. 1-chloro-2-(3-(trifluoromethyl) phenyl) diazene (4) in-situ with isopropenyl acetate (3) to obtain 1-(3-(trifluoromethylphenyl) propan-2-one (2);
iii. aminating 1-(3-(trifluoromethylphenyl) propan-2-one (2) with ethyl amine to obtain Fenfluramine hydrochloride crude (1a); and
iv. purifying crude Fenfluramine hydrochloride (1a) to obtain Fenfluramine hydrochloride (1).

In second aspect, embodiments of the present invention provides process for the purification of Fenfluramine hydrochloride (1), which is substantially free of Impurity A and Impurity B comprises:
a) dissolving Fenfluramine hydrochloride crude (1a) in a suitable solvent;
b) cooling to suitable temperature; and
c) isolating Fenfluramine hydrochloride (1).

In third aspect, embodiments of the present invention provides process for the preparation of 3-trifluoromethylaniline hydrochloride (5) having purity greater than 99.5%, which comprises:
1. nitrating benzotrifluoride (7) with nitric acid in the presence of sulfuric acid to obtain 3-nitrobenzotrifluoride (6);
2. reducing 3-nitrobenzotrifluoride (6) in presence of reducing agent to obtain crude 3-trifluoromethylaniline hydrochloride (5a); and
3. purifying crude 3-trifluoromethylaniline hydrochloride (5a) to obtain 3-trifluoromethylaniline hydrochloride (5).

In fourth aspect, embodiments of the present invention provides process for the purification of 3-trifluoromethylaniline hydrochloride (5), which is substantially free of Impurity C and Impurity D comprises.

A. dissolving crude 3-trifluoromethylaniline (5a) in a suitable solvent;

B. adding hydrochloric acid to step A);

C. dissolving 3-trifluoromethylaniline hydrochloride in suitable solvent;
D. cooling to suitable temperature; and
E. isolating 3-trifluormethylaniline hydrochloride (5).

Embodiments of the present invention provides process for the preparation of substantially pure 3-trifluoromethylaniline hydrochloride (5), intermediate which is being used as starting material to obtain pure Fenfluramine hydrochloride (1). Using substantially pure starting materials is advantages to control yield loss in the purification of final product.

### BRIEF DESCRIPTION

Some of the embodiments will be described in detail, with references to the following Figures, wherein like designations denote like members, wherein:

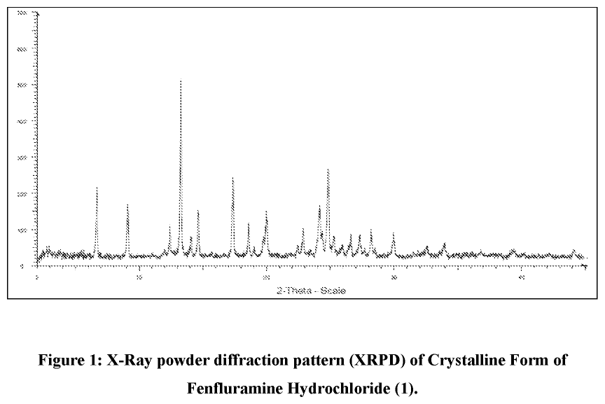

FIG. 1 illustrates X-Ray Diffraction (XRD) pattern Fenfluramine hydrochloride (1); and

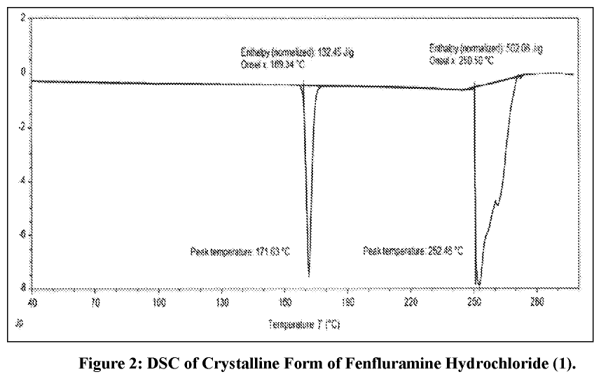

FIG. 2 illustrates DSC of Fenfluramine hydrochloride (1).

### DETAILED DESCRIPTION

In one embodiment, the present invention provides an improved process for the preparation of Fenfluramine hydrochloride (1) having purity greater than 99.5% by HPLC using highly pure 3-(trifluoromethyl) aniline hydrochloride (5) as a key starting material, which is illustrated in scheme 1:

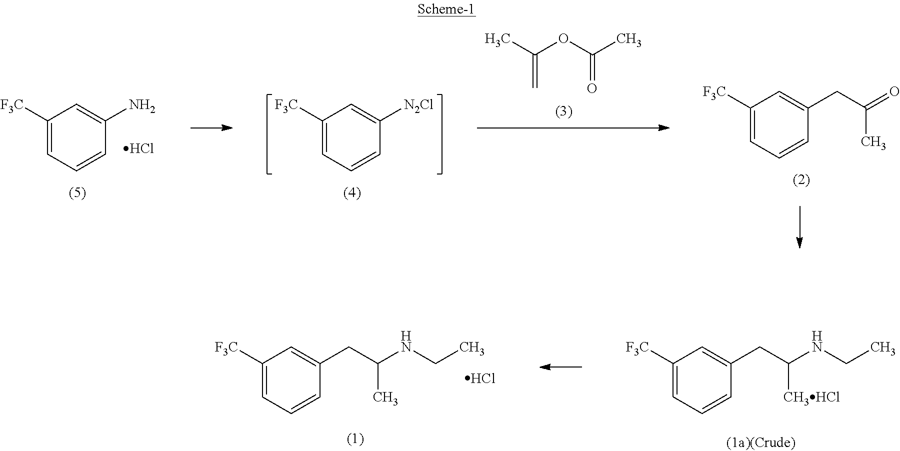

The process comprises:
Step i) involves reacting 3-trifluoromethylaniline hydrochloride (5) with sodium nitrite or nitrous acid in the presence of acid to obtain 1-chloro-2-(3-(trifluoromethyl) phenyl) diazene (4). The acid used in step (i) is selected from hydrochloric acid, sulfuric acid desirably using hydrochloric acid.
Step ii) involves 1-chloro-2-(3-(trifluoromethyl) phenyl) diazene (4) in-situ with isopropenyl acetate (3) in presence of reducing agent in a suitable solvent, at a temperature of 40-50° C. to obtain 1-(3-(trifluoromethylphenyl) propan-2-one (2).
Step iii) involves aminating 1-(3-(trifluoromethylphenyl) propan-2-one (2) with ethyl amine in the presence of reducing agent in a suitable solvent to obtain Fenfluramine hydrochloride crude (1a).
Step iv) involves purifying crude Fenfluramine hydrochloride (1a) in a suitable solvent followed by cooling to suitable temperature and isolate Fenfluramine hydrochloride (1).

In second embodiment, the present invention provides process for the purification of Fenfluramine hydrochloride (1), which is substantially free of Impurity A and Impurity B. The process comprises:
a) dissolving crude Fenfluramine hydrochloride (1a) in a suitable solvent;
b) cooling to suitable temperature; and
c) isolating Fenfluramine hydrochloride (1).

The free of impurities in Fenfluramine hydrochloride (1) includes 2-Fenfluramine (Impurity A) and 4-Fenfluramine (Impurity B).

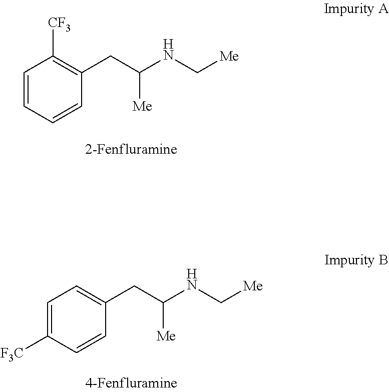 [SMILES: CCNC(C)Cc1ccccc1C(F)(F)F ; CCNC(C)Cc1ccc(C(F)(F)F)cc1]

In third embodiment, the present invention provides process for the preparation of 3-trifluoromethylaniline hydrochloride (5) is having purity greater than 99.5%, which is illustrated in scheme 2:

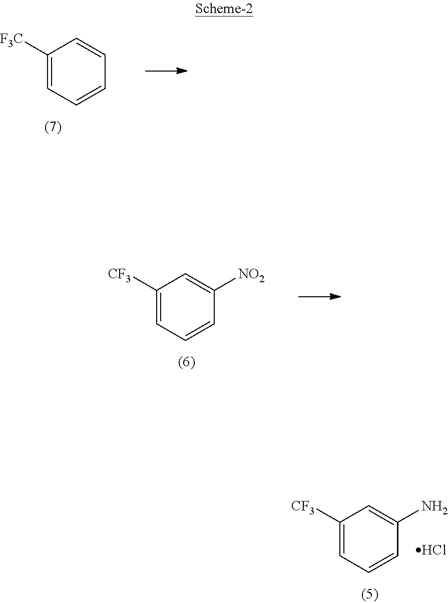 [SMILES: FC(F)(F)c1ccccc1 ; O=[N+]([O-])c1cccc(C(F)(F)F)c1 ; Nc1cccc(C(F)(F)F)c1 ; Cl]

The process comprises:
Step 1): nitrating benzotrifluoride (7) with nitric acid in the presence of sulfuric acid to obtain 3-nitrobenzotrifluoride (6);
Step 2) reducing 3-nitrobenzotrifluoride (6) in presence of Raney nickel in methanol and hydrochloric acid to obtain crude 3-trifluoromethylaniline hydrochloride (5); and
Step 3) purifying crude 3-trifluoromethylaniline hydrochloride (5a) to obtain 3-trifluoromethylaniline hydrochloride (5).

In fourth embodiment, the present invention provides process for the purification of 3-trifluoromethylaniline hydrochloride (5), which is substantially free of Impurity C and Impurity D. The process comprises:
A. dissolving 3-trifluoromethylaniline (5a) in a suitable solvent;
B. adding hydrochloric acid to step A);
C. dissolving 3-trifluoromethylaniline hydrochloride in suitable solvent;
D. cooling to suitable temperature; and
E. isolating 3-trifluormethylaniline hydrochloride (5).

The free of impurities in 3-trifluoromethylaniline hydrochloride (5) includes 2-(trifluoromethyl) aniline (Impurity C) and 4-(trifluoromethyl) aniline (Impurity D).

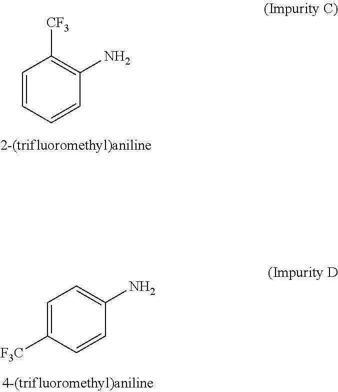 [SMILES: Nc1ccccc1C(F)(F)F ; Nc1ccc(C(F)(F)F)cc1]

The term “reducing agents” used in embodiments of the present invention is selected from cuprous chloride, cupric chloride dihydrate, tin chloride, iron powder, sodium acetoxy borohydride, sodium borohydride, Raney nickel, palladium carbon, sodium cyanoborohydride, lithium aluminium hydride, desirably cuprous chloride, sodium acetoxy borohydride and Raney Nickel.

The term “suitable solvent” used in embodiments of the present invention is selected from water, ethanol, methanol, dichloromethane, acetonitrile, dioxane, dimethyl sulfoxide, isopropanol, acetone, ethyl acetate, benzene, toluene, heptane, xylene, methylene chloride, chloroform, dioxane, tetrahydrofuran, anisole, N,N-dimethylformamide, N,N-dimethyl acetamide and the like or mixtures thereof, desirably water, methanol, ethanol, ethyl acetate, heptane.

In fifth embodiment, 3-trifluormethylaniline hydrochloride (5) obtained according to embodiments of the present invention is having purity greater than 99.5% by HPLC with total impurities (Nitro impurity and unknown impurities) of less than 5% (w/w), less than 3% (w/w), or less than 0.15% (w/w).

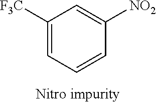 [SMILES: O=[N+]([O-])c1cccc(C(F)(F)F)c1]

In sixth embodiment, Fenfluramine hydrochloride obtained according to embodiments of the present invention has total impurities comprising of Norfenfluramine, Keto impurity, and Alcohol impurity less than 5% (w/w), or less than 3% (w/w), or less than 0.15% (w/w).

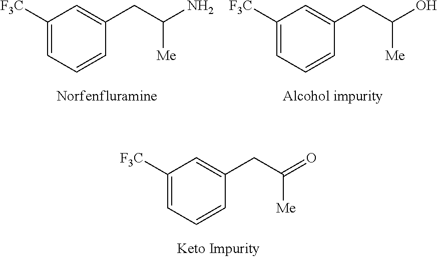 [SMILES: CC(N)Cc1cccc(C(F)(F)F)c1 ; CC(O)Cc1cccc(C(F)(F)F)c1 ; CC(=O)Cc1cccc(C(F)(F)F)c1]

In seventh embodiment, Fenfluramine hydrochloride obtained according to embodiments of the present invention has water content less than 3% (w/w), or less than 1% (w/w).

In eighth embodiment, crystalline form of Fenfluramine hydrochloride obtained according to embodiments of the present invention is characterised by X-ray powder diffractions (XRD) pattern as shown in FIGS. 1 and 2 theta values as provided in Table 1:

TABLE 1

| Two theta (2Θ) values | Relative Intensity I/Io (%) |
|---|---|
| 6.6 | 42.5 |
| 9.0 | 32.5 |
| 12.3 | 21.6 |
| 13.2 | 100 |
| 14.0 | 15 |
| 14.6 | 29.4 |
| 17.3 | 47.1 |
| 18.5 | 22.7 |
| 19.0 | 9.9 |
| 19.7 | 14.3 |
| 19.9 | 29.3 |
| 22.4 | 10.4 |
| 22.8 | 19.8 |
| 24.1 | 32.2 |
| 24.3 | 17.2 |
| 24.8 | 52.1 |
| 25.2 | 15.9 |
| 25.9 | 11.4 |
| 26.5 | 16.4 |
| 27.3 | 15.7 |
| 28.2 | 19.4 |
| 29.9 | 17.2 |

The following examples further illustrate embodiments of the present invention but should not be construed in any way as to limit its scope.

### EXAMPLES

### Example-1: Process for the Preparation of 1-(3-(trifluoromethylphenyl) propan-2-one (2)

To 20 gm of 3-(trifluoromethyl) aniline hydrochloride (5), water and 30 ml of HCl was added at 0-5° C., followed by dropwise addition of sodium nitrite to form 1-chloro-2-(3-(trifluoromethyl) phenyl) diazene (4) in-situ. In another reaction flask methanol, copper chloride, anhydrous sodium acetate and isopropenyl acetate (3) was added and stirred at 40-45° C. To the reaction mass 1-chloro-2-(3-(trifluoromethyl) phenyl) diazene (4) was added dropwise, stirred, and cooled to 25-30° C. 50 ml of heptane was added to the reaction mass, organic and aqueous layers were separated. To the organic layer sodium metabisulfite was added and stirred to obtain the reaction mass. The reaction mass was filtered and 60 ml of mixture of heptane and water was added. The reaction mass was cooled, and 30 ml of sodium hydroxide solution was added, stirred and the layers were separated. The organic layers were washed and distilled off to obtain 1-(3-(trifluoromethylphenyl) propan-2-one (2). Yield: 50%; Purity: 85%.

### Example-2: Process for the Preparation of Fenfluramine Hydrochloride (1a)

20 gm of 1-(3-(trifluoromethylphenyl) propan-2-one (2) was dissolved in methanol at 25-30° C. and cooled to 10-15° C. To the reaction mass 25 ml of ethylamine was added dropwise and stirred. 50 gm of sodium triacetoxy borohydride was added to the reaction mass and stirred at 25-30° C., pH was adjusted to 10-12 using 20% sodium hydroxide solution. 50 ml of toluene was added to the reaction mass and layers were separated. The organic layers were combined and washed with water and distilled at 40-50° C. To the reaction mass water and toluene was added and cooled to 10-15° C. and pH was adjusted to 0.5-2.0 using concentrated hydrochloric acid. The aqueous layer was adjusted to pH 10-12 using sodium hydroxide and 1 vol ethyl acetate was added and stirred at 25-30° C. The organic layer was dried over sodium sulphate at 25-30° C., distilled at 40-50° C. and degassing for 20 min. To the crude obtained 2 vol of ethyl acetate was added and cooled to 0-5° C., ethyl acetate hydrochloride was added and stirred at 35-40° C. The reaction mass was filtered and dried to obtain Fenfluramine hydrochloride crude (1a). Yield: 80%.

### Example-3: Purification of Fenfluramine Hydrochloride (1)

20 gm of crude Fenfluramine hydrochloride (1a) was dissolved in 40 ml of methanol and stirred at 25-30° C. 30 ml of ethyl acetate was added and heated to 60-65° C., the reaction mass cooled to 5-10° C. and stirred for 1 hour. The obtained solid was filtered, and dried to obtain fenfluramine hydrochloride (1). Yield: 70%; Purity: 99.98%; XRD: FIG. 1: DSC: FIG. 2

### Example-4: Purification of 3-(trifluoromethyl) aniline hydrochloride (5)

20 gm of 3-(trifluoromethyl) aniline hydrochloride (5a) was dissolved in water, 30 ml of hydrochloric acid was added and heated to 25-30° C. To the reaction mass methanol was added and heated to 50-55° C., ethyl acetate was added, stirred and cooled to 25-30° C., filtered, washed with ethyl acetate to obtain 3-(trifluoromethyl) aniline hydrochloride (5). Yield: 87.2%; Purity: 99.98%.

Although the invention has been illustrated and described in greater detail with reference to the preferred exemplary embodiments, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1-11. (canceled)

12. An improved process for the preparation of fenfluramine hydrochloride 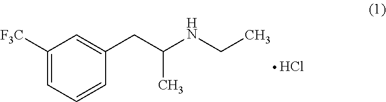 [SMILES: CCNC(C)Cc1cccc(C(F)(F)F)c1 ; Cl]
comprising:
i. reacting 3-trifluoromethylaniline hydrochloride 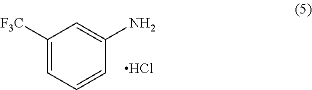 [SMILES: Nc1cccc(C(F)(F)F)c1 ; Cl]
with sodium nitrite or nitrous acid to obtain 1-chloro-2-(3-(trifluoromethyl) phenyl) diazene, 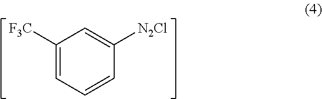
ii. the compound of formula in-situ with isopropenyl acetate 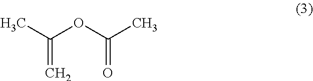
to obtain 1-(3-(trifluoromethylphenyl) propan-2-one, 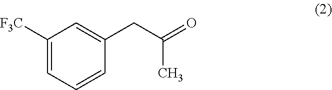
iii. aminating the compound of formula with ethyl amine to obtain fenfluramine hydrochloride crude, and
iv. purifying crude fenfluramine hydrochloride to obtain fenfluramine hydrochloride,
wherein the fenfluramine hydrochloride obtained is substantially free of 2-fenfluramine (Impurity A) and 4-fenfluramine (Impurity B) impurities.

13. The process as claimed in claim 12, wherein purifying fenfluramine hydrochloride, comprising:
a) dissolving crude fenfluramine hydrochloride in a suitable solvent;
b) cooling to suitable temperature; and
c) isolating fenfluramine hydrochloride.

14. A process for purifying 3-trifluoromethylaniline hydrochloride, comprising:
A. dissolving crude 3-trifluoromethylaniline in a suitable solvent;
B. adding hydrochloric acid to the reaction mass;
C. dissolving 3-trifluoromethylaniline hydrochloride in suitable solvent;
D. cooling to suitable temperature; and
E. isolating 3-trifluormethylaniline hydrochloride.

14. The process as claimed in claim 12, wherein the reaction is carried out in a solvent selected from water, ethanol, methanol, dichloromethane, acetonitrile, dioxane, dimethyl sulfoxide, isopropanol, acetone, ethyl acetate, benzene, toluene, heptane, xylene, methylene chloride, chloroform, dioxane, tetrahydrofuran, anisole, N, N-dimethylformamide, N, N-dimethyl acetamide and the like or mixtures thereof.

15. The process as claimed in claim 13, wherein the reaction is carried out in a solvent selected from water, ethanol, methanol, dichloromethane, acetonitrile, dioxane, dimethyl sulfoxide, isopropanol, acetone, ethyl acetate, benzene, toluene, heptane, xylene, methylene chloride, chloroform, dioxane, tetrahydrofuran, anisole, N, N-dimethylformamide, N, N-dimethyl acetamide and the like or mixtures thereof.

16. The process as claimed in claim 14, wherein the reaction is carried out in a solvent selected from water, ethanol, methanol, dichloromethane, acetonitrile, dioxane, dimethyl sulfoxide, isopropanol, acetone, ethyl acetate, benzene, toluene, heptane, xylene, methylene chloride, chloroform, dioxane, tetrahydrofuran, anisole, N, N-dimethylformamide, N, N-dimethyl acetamide and the like or mixtures thereof.

17. The process as claimed in claim 12, wherein the reaction is carried out in step-i) in presence of inorganic acid selected from hydrochloric acid, hydrobromic acid, sulfuric acid, nitric acid, and phosphoric acid.

18. The process as claimed in claim 12, wherein the reducing agent used in step-ii) and step-iii) is selected from cuprous chloride, cupric chloride dihydrate, tin chloride, iron powder, sodium acetoxy borohydride, sodium borohydride, Raney nickel, palladium carbon, sodium cyanoborohydride and lithium aluminium hydride.

19. The process as claimed in claim 12, wherein fenfluramine hydrochloride is having norfenfluramine, keto impurity, and alcohol impurity less than 0.15%.

20. The process as claimed in claim 12, wherein 3-trifluoromethylaniline hydrochloride is having nitro impurity less than 0.15%.

21. A compound fenfluramine hydrochloride is having a purity of at least 99.5% by HPLC, wherein fenfluramine hydrochloride or its free base is free of 2-fenfluramine (impurity A) and 4-fenfluramine (impurity B) impurities and having norfenfluramine, keto impurity, alcohol impurity less than 0.15%, and moisture content less than 3.0% (w/w).
